(19) United States
(12) Patent Application Publication
(10) Pub. No.: US 2022/0160389 A1
(43) Pub. Date: May 26, 2022

(54) ENDOLUMINAL PUNCH SYSTEM

(71) Applicant: Indian Wells Medical, Inc., Lake Forest, CA (US)

(72) Inventors: Jay Alan Lenker, Lake Forest, CA (US); James Alexander Carroll, Long Beach, CA (US); Eugene Michael Breznock, Winters, CA (US); Donald J. Kolehmainen, Laguna Niguel, CA (US)

(73) Assignee: Indian Wells Medical, Inc., Lake Forest, CA (US)

(21) Appl. No.: 17/534,288

(22) Filed: Nov. 23, 2021

Related U.S. Application Data
(60) Provisional application No. 63/118,523, filed on Nov. 25, 2020.

(51) Int. Cl. A61B 17/3205; A61M 29/00

(52) U.S. Cl. CPC A61B 17/32053; A61M 29/00

(57) ABSTRACT

An endoluminal punch and introducer sheath. The endoluminal punch includes a secondary blade, or fin blade affixed to the distal end of the endoluminal punch to increase the size of an incision.

### FIELD OF THE INVENTION

The invention relates to devices and methods for performing endovascular access to the cardiovascular system or other body vessels or body lumens, especially procedures performed in the fields of cardiology, radiology, electrophysiology, and surgery.

### BACKGROUND

The currently accepted procedure for left atrial access involves routing a needle called a Brockenbrough needle into the right atrium with the Brockenbrough needle pre-placed within a guiding catheter. The guiding catheter specifically preferred for use with a Brockenbrough needle is called a Mullins-type catheter or transseptal introducer. The Brockenbrough needle is a long, small diameter punch, generally formed from a stainless steel wire stylet that is surrounded by a stainless steel tube. Other devices, designed for the same purpose, employ radiofrequency ablation to perforate the atrial wall but these devices expose the myocardium to burning, potentially reduced healing characteristics, and increased risk of subsequent scarring.

### SUMMARY

The methods and devices disclosed below provide for easier penetration of a dilator of an endoluminal punch system through the wall of a body lumen, such as the fossa ovalis. The endoluminal punch of the system is fitted with a radially extending cutting blade configured to cut a portion of a perforation in the tissue initially created by the penetrating tip of the punch, to make it easier for the larger dilator tip surrounding the punch to pass through the perforation. The dilator has, in addition to the lumen which accommodates the main portion of the punch (typically a round lumen to accommodate a tubular or cylindrical punch with a round transverse cross section), the dilator has a channel, open to the lumen and extending the length of the dilator lumen, sized to accommodate the cutting blade extending radially from the punch. In use, as the punch is advanced out the distal end of the dilator to penetrate the fossa ovalis, the cutting blade with extend radially from a tapered distal tip of the dilator to engage the fossa ovalis and cut a small slit in the fossa ovalis at the edge of the perforation, facilitating passage of the dilator through the perforation.

### BRIEF DESCRIPTION OF THE DRAWINGS

FIG. 1 illustrates a steerable endoluminal punch, as viewed from the side in cross-section, whereby the steerable endoluminal punch comprises a vibratory actuator operably connecting the primary hub structure to the jackscrew holding portion of the hub, according to an embodiment of the invention.

FIG. 2 illustrates a steerable endoluminal punch, as viewed from the side in cross section, whereby a vibratory actuator is operably connected to the proximal end of the hub of the steerable endoluminal punch.

FIG. 3A illustrates an introducer further comprising a dilator, wherein the dilator comprises a cutting element shown in its retracted configuration within the main lumen, according to an embodiment of the invention.

FIG. 3B illustrates the introducer of FIG. 3A wherein an endoluminal punch, steerable or otherwise, has been inserted through the main lumen of the dilator causing the cutting element to be forced out of the central lumen so as to project laterally outside the tapered tip of the dilator, according to an embodiment of the invention.

FIG. 4 illustrates an introducer, further comprising a dilator in side partial breakaway view, whereby the dilator comprises a central lumen and a side lumen for passage of instrumentation, linkages, and the like, according to an embodiment of the invention,

FIG. 5A upper illustrates a hole poked through a piece of tissue and FIG. 5A bottom illustrates that same hole dilated to 0.032 diameter by the tip diameter of a steerable endoluminal punch.

FIG. 5B upper illustrates a semicircular slot cut through a piece of tissue by an endoluminal punch with circumferential blade and FIG. 5B bottom shows the same semicircular slot dilated to its maximum potential diameter with no tissue stretching, according to an embodiment of the invention.

FIG. 5C upper illustrates a semicircular slot cut through a piece of tissue and an additional slit created by the cutting element of an introducer dilator as illustrated in FIG. 3B while FIG. 5C bottom shows the same slot dilated to its maximum circular diameter with no tissue stretching, according to an embodiment of the invention.

FIG. 6A illustrates a side view, in partial breakaway of the distal end of an introducer comprising a sheath and a dilator, wherein the dilator comprises an internal lumen with a radial projection that passes the entire length of the internal lumen except for a stop at the distal end, according to an embodiment of the invention.

FIG. 6B illustrates an axial view, toward the distal end of the introducer of FIG. 6A, wherein the lumen radial projection stop is visible, according to an embodiment of the invention.

FIG. 6C illustrates a side view, in partial breakaway of the distal end of an introducer comprising a sheath and a dilator, wherein the dilator comprises an internal lumen with a radial projection that passes the entire length of the internal lumen, according to an embodiment of the invention.

FIG. 6D illustrates an axial view, toward the distal end of the introducer of FIG. 6C, wherein the dilator does not comprise a lumen radial projection stop, according to an embodiment of the invention.

FIG. 7A illustrates a side view, in partial breakaway, of the distal end of the introducer of FIG. 6A, further comprising a transseptal needle having a radially projecting cutting blade, according to an embodiment of the invention.

FIG. 7B illustrates a side view, in partial breakaway, of the distal end of the introducer of FIG. 6C, further comprising a transseptal needle having a radially projecting cutting blade, according to an embodiment of the invention.

FIG. 8A illustrates the distal end of a transseptal needle front end and side view, comprising a radially projecting cutting blade that extends a short distance distally from a stepdown, according to an embodiment of the invention;

FIG. 8B illustrates the distal end of a transseptal needle front end and side view, comprising a radially projecting cutting blade that extends a longer distance distally from a stepdown, according to an embodiment of the invention.

FIG. 9A illustrates a side view of the distal end of an endoluminal punch inserted through an introducer, wherein the endoluminal punch further includes a piercing stylet or center punch having a flattened distal end further enhanced by a lateral deflection and a sharp cutting edge along the front and top surfaces of the piercing stylet, according to an embodiment of the invention.

FIG. 9B illustrates a side view of the distal end of an endoluminal punch inserted through an introducer, wherein the endoluminal punch further includes a piercing stylet or center punch having a flattened distal end further enhanced by a lateral deflection and a sharp cutting edge along the bottom and front surfaces of the piercing stylet, according to an embodiment of the invention.

### DETAILED DESCRIPTION

In accordance with current terminology pertaining to medical devices, the proximal direction will be that direction on the device that is furthest from the patient and closest to the user, while the distal direction is that direction closest to the patient and furthest from the user. These directions are applied along the longitudinal axis of the device, which is generally an axially elongate structure having one or more lumens or channels extending through the proximal end to the distal end and running substantially the entire length of the device.

In an embodiment, the invention is an endoluminally, transvascularly, or endovascularly placed tissue punch, with internal deflectability or the ability to articulate, at its distal end, in a direction away from its longitudinal axis. The punch can also be termed a catheter, needle, or cannula. The punch is generally fabricated from stainless steel and comprises an outer tube, an intermediate tube, a central stylet wire, and a distal articulating region. The deflecting or articulating mechanism is integral to the punch. The punch, needle, or catheter is sufficiently rigid, in an embodiment, that it can be used as an internal guidewire or internal guide catheter. The punch is useful for animals, including mammals and human patients and is routed through body lumens or other body structures to reach its target destination.

In an embodiment, the punch comprises a core wire or stylet, an inner tube and an outer tube. The inner tube can comprise a sharpened distal end to facilitate tissue puncture. The sharpened end can comprise bevels, facets, sharpened blade-like structures, or the like. The core wire or stylet can be blunted at the distal end to prevent damage to structures such as tissue, the sheath, or the dilator during advancement of the endoluminal punch, caused by the sharpened distal end of the endoluminal punch. In an embodiment, the stylet can be removable or non-removable. In some embodiments, the stylet can have a large diameter to minimize trauma and shield sharp structures on the distal tip of the endoluminal punch. The endoluminal punch further comprises a hub at its proximal end which permits grasping of the punch and can also include a stopcock or valve to serve as a lock for the stylet, as well as a valve for control of fluid passage into and out from the innermost lumen within which the stylet or inner core wire resides. The hub can further comprise additional ports to facilitate the administration or withdrawal of fluids or pressure measurement. The additional ports can be terminated with Luer lock connectors or with flexible lead lines terminated with Luer lock connectors, stopcocks, or the like. The proximal end further can comprise one or more control mechanisms to manipulate the amount of articulation at the distal end of the catheter. The proximal end further is terminated with a female Luer or Luer lock port, which is suitable for attachment of pressure monitoring lines, dye injection lines, vacuum lines, a combination thereof, or the like. Other structures can be provided to alter the distal tip of the endoluminal punch such as changing it from blunter and less traumatic to sharper and more capable of tissue penetration. Such distal tip altering structures can include a piercing stylet which has an extremely sharp distal end or which transmits energy to the distal tip of the endoluminal punch. The energy can be in the form of simple manually applied force, mechanical vibration, mechanical rotation, ultrasound, high intensity focused ultrasound, electrical power to heat the distal tip, cryogenic energy, laser energy, and the like. The distal tip altering structure can comprise a quick release or controlled retraction mechanism which can be dumb or it can be responsive to measurements of force, tissue properties, or the like.

Other embodiments of the inventions comprise methods of use. Keeping the method of use as close to current techniques is preferable since it reduces the learning curve and physician confidence in the procedure. The general procedure comprises placing a guidewire beyond the right atrium via a percutaneous access point in the right femoral vein or jugular vein. A transseptal introducer is advanced over the guidewire, the transseptal introducer comprising a sheath and a dilator. The dilator further comprises a shaft, a tapered distal tip, a central through lumen, and a hub affixed to the proximal end of the shaft. The sheath comprises a hemostasis valve to seal to the dilator shaft, a side port with stopcock communicating with the central lumen of the sheath, and the like. The sheath and the dilator can comprise a pre-formed curve near the distal end. The guidewire is next removed and a transseptal needle or other crossing system is advanced through the central lumen of the dilator. The transseptal needle with the transseptal introducer riding on its back can be targeted at a specific site on the interatrial wall, generally in the area of the fossa Ovalis. The tissue is tented by the dilator to stretch the tissue at the target site and exert a crossing force on the tissue. The transseptal needle is preferably retracted within the blunt distal tip of the dilator to prevent any chance of unwanted or inadvertent tissue perforation. Once the target is secured, the transseptal needle is advanced distal to the distal tip of the dilator thus exposing it to the tissue and causing cutting of the tented tissue. The transseptal needle and dilator/sheath are advanced across the tissue to gain access to the other side. The transseptal needle and dilator can be removed at this time to provide a pathway through the sheath or a guidewire can be reinserted to provide a track for subsequent catheterizations.

In another embodiment, the core wire, stylet is sharpened and serves as a tissue punch. In this embodiment, the distal end of the hollow tubes of the punch are blunted and made relatively atraumatic. Once the core wire punch has completed tissue penetration, the outer tubes are advanced over the central punch wire through the penetration and into the left atrium. In another embodiment, a pressure monitoring device such as a catheter tip pressure transducer, or a pressure line terminated by a pressure transducer, can be affixed to a quick connect, generally a Luer fitting, at the proximal end of the punch hub. By monitoring pressure, it is possible to determine when the distal end of the punch has passed from, for example, the right atrium into the left atrium, because the pressure versus time curves in these two chambers are measurably, or visually, different. The proximal end of the hub further has provision for attachment to a dye injection line for use in injecting radiographic contrast media through the central lumen of the punch. Typically, a manifold can be attached to the Luer fitting on the proximal end of the hub, the manifold allowing for pressure monitoring, for example on a straight through port, and for radiopaque dye injection, for example through a side port. A stopcock, or other valve, can be used to control which port is operably connected to the central lumen of the punch.

In some embodiments, the inner tube, the outer tube, or both can have slots imparted into their walls to impart controlled degrees of flexibility. The slots can be configured as “snake cuts” to form a series of ribs with one or more spines. The spines can be oriented at a given circumferential position on the outer tube, the inner tube, or both. The spines can also have non-constant orientations. In some embodiments, only the outer tube is slotted. The slots can be generated within the distal portion of the outer tube where the curve is generated. This bendable distance can range between about 0.5-cm and 20-cm of the end and preferably between about 1-cm and 12-cm of the distal end. The slot widths can range between 0.001 inches and 0.010 inches with a preferable width of about 0.001 to 0.005 inches. In exemplary embodiments, the slot widths are about 0.003 inches. In some embodiments, it is desirable to have the outer tube bend in one direction only but not in the opposite direction and not in either lateral direction. In this embodiment, cuts can be made on one side of the outer tubing within, for example, the distal 10-cm of the tube length. Approximately 10 to 30 cuts can be generated with a width of approximately 0.001 to 0.015 inches. The cut depth, across the tube diameter from one side, can range between 0.01 and 0.9 of the tube's diameter. In an embodiment, the cut depth can be approximately 0.4 to 0.6 of the tube's diameter with a cut width of about 0.005 inches or less. A second cut can be generated on the opposite side of the tube wherein the second cut is approximately 0.005 inches or less. In an embodiment, the outer tube can be bent into an arc first and then have the slots generated such that when the tube is bent back toward the 0.005-inch wide cuts, the tube will have an approximately straight configuration even through each tube segment between the cuts is slightly arced or curved.

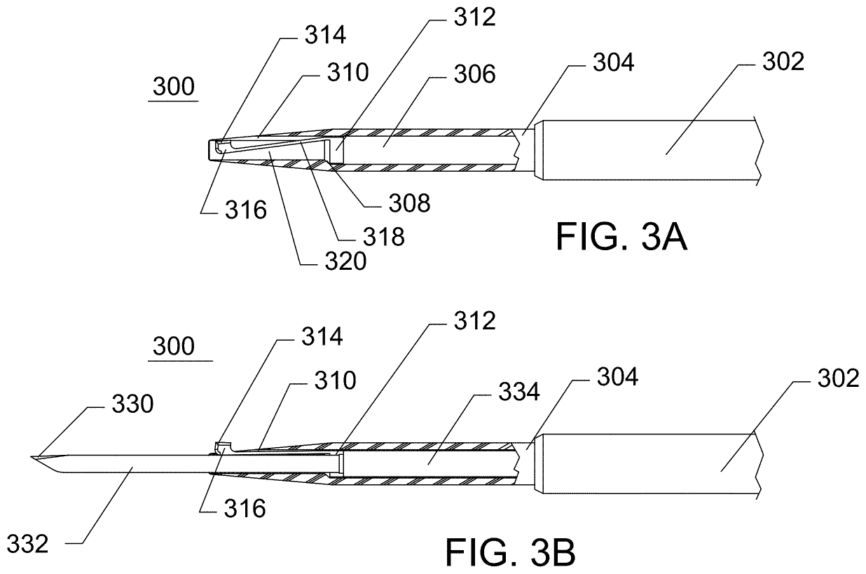

FIG. 3 illustrates a cutting introducer 5630 further comprising the introducer sheath 5602, a dilator 5624 further comprising a cutting element lumen 5622 and a cutting element 5620. An endoluminal punch 5610 is inserted into a central lumen of the dilator 5624. The optional piercing stylet 5616 is inserted through a central lumen of the endoluminal punch 5610. Distal end of the larger diameter portion of the endoluminal punch 5610 is configured to engage the proximal end of the cutting element 5620 and force it distally thus exposing its distal sharp end to assist with tissue incision. The cutting element 5620 is trapped within the cutting element lumen 5622 and cannot be removed from the system. The cutting element 5620 is preferably biased in its retracted position by a spring (not shown) or other biasing element.

The steerable needle, in other embodiments, can comprise monitoring systems to measure, display, announce, record, or evaluate operating parameters of the steerable transseptal needle. In an embodiment, the steerable transseptal needle can comprise strain gauges to measure the force being applied by the user to bend the needle. A torque gauge can also be comprised by the system to measure torque being applied to the control knob or the torque being applied by the distal curvature movement. The strain gauge or torque gauge can be affixed within the hub or elsewhere within the steerable transseptal needle to measure compression or tension forces. This information can be displayed in the form of a readout device, such as a digital display of the force or torque. The number of turns can be counted and displayed by, for example, a Hall-Effect sensor, mechanical counter, or the like. In an embodiment, the force or toque can be correlated to the angle of deflection at the distal end, the number of turns applied to the control knob, or both. The readout can be digital or analog and can be affixed to the hub or can be wirelessly received and displayed on external equipment such as a smart phone, computer, tablet computer, panel display, or the like. The wireless technology can, for example, comprise Wi-Fi, Bluetooth®, or other standardized protocols. The human interface can, in other embodiments, comprise audible feedback such as a simple beep or tone, or it can be more sophisticated and provide information using language callouts such as force, turns, torque, or the like.

In operation, the procedure is to advance a steerable transseptal needle, with a tissue piercing stylet affixed in place, through a transseptal introducer that has already been placed. The steerable transseptal needle is articulated to generate the proper curve, as determined under fluoroscopic or ultrasound guidance. The steerable transseptal needle transseptal introducer assembly is withdrawn caudally out of the superior vena cava and into the right atrium of the heart. Proper location, orientation, tenting, and other features are confirmed. Radiopaque dye can be injected through the steerable transseptal needle to facilitate marking of the fossa ovalis or blood flow around the distal end of the steerable transseptal needle. Pressure measurements can also be taken through the lumen of the steerable transseptal needle to confirm tracings consistent with the right or left atrium of the heart. Once proper positioning has been confirmed, a safety is removed from the stylet hub and a button on the stylet hub is depressed or actuated to cause the sharpened stylet tip to advance out beyond the distal end of the steerable transseptal needle. This sharpened stylet punches through the fossa ovalis and the septal tissue pulls over the stylet, over the inner tube, and over the dilator of the transseptal introducer. At this point, the sharp stylet is released and retracts proximally within the steerable transseptal needle. The transseptal introducer is now within the left atrium of the heart and the steerable transseptal introducer can be withdrawn from the lumen of the dilator.

FIG. 3A illustrates another embodiment of a dilator and introducer for a steerable endoluminal punch. The introducer 300 comprises an introducer shaft 302, having a tapered or faired tip and a dilator 304, further comprising a central lumen 306, an internal stepdown 308, to serve as a stop for an endoluminal punch, a small diameter tip lumen 320, a radially outwardly directed slit or fenestration 310, a cutting element 316 disposed within the central lumen 306, a spring arm 318, and a tip cutter 316 having a sharp edge 314.

The side slot 310 communicates with the tip central lumen 320 and permits the cutting element 316 and its spring arm 318 to move radially outward beyond the central lumen 320, wherein it resides when at rest, biased by the spring arm 318. The cutting element 316 can be fabricated from stainless steel, nitinol, titanium, cobalt nickel alloy or the like.

FIG. 3B illustrates the introducer and dilator of FIG. 3A with an endoluminal punch 334 having been inserted through the central lumen 306 and 320 as far as possible with a stepdown on the endoluminal punch engaging the stepdown in the lumen 308. A small diameter portion of the endoluminal punch 332 projects through the lumen 320 and forces the spring arm 318 and the cutting element 316 radially outward through the slot 310. The sharp edges 314 are configured to cut tissue as the dilator is advance through tissue thus allowing a larger diameter incision to be made than could be made with the endoluminal punch, by itself.

In this embodiment, the endoluminal punch 334 can be inserted through the introducer 300 further comprising the dilator 304 which can further comprise the cutting apparatus 316 tipped with 314, which can project laterally to assist with cutting tissue surrounding the tapered tip of the dilator. The cutting apparatus can comprise a sharp blade like structure. The cutting apparatus can be spring loaded so that, when something is advanced through the internal lumen of the dilator, the blade is forced out of the way and projects laterally outward. This way, the lateral cutting blade is restricted inside the dilator partially or completely when nothing is inserted through the lumen. A segment of the lateral cutting blade can be dispositioned within the lumen 320 to facilitate outward advance of the cutting blade or apparatus when another object is forced through the lumen. This feature can be useful in penetrating tissue with the Steerable Endoluminal Punch and then following it with the larger diameter dilator and introducer or catheter. The laterally projecting blade or cutter can enlarge a hole in tissue already incised by the Endoluminal Punch, thus facilitating passage of the dilator and introducer through the hole in the tissue. Conditions can occur in patients who have had prior procedures and therefore incur scar tissue on the interatrial septum, for example, and this tissue is very difficult to cross. Further, it appears that patients who have been in long term persistent atrial fibrillation incur morphological changes to the myocardium in the interatrial septum, and perhaps other parts of the atrial tissue, that cause it to become tough to penetrate.

In other embodiments, the steerable endoluminal punch can comprise a blunted distal end with a slot at the end to allow a blade to project out the distal end of the steerable endoluminal punch. The blunted distal end can be retracted to expose the blade for cutting or the blade can be advanced out the distal end through the slot which would appear like the window of an observatory. The blade can be fixed or it can oscillate or rotate as described herein.

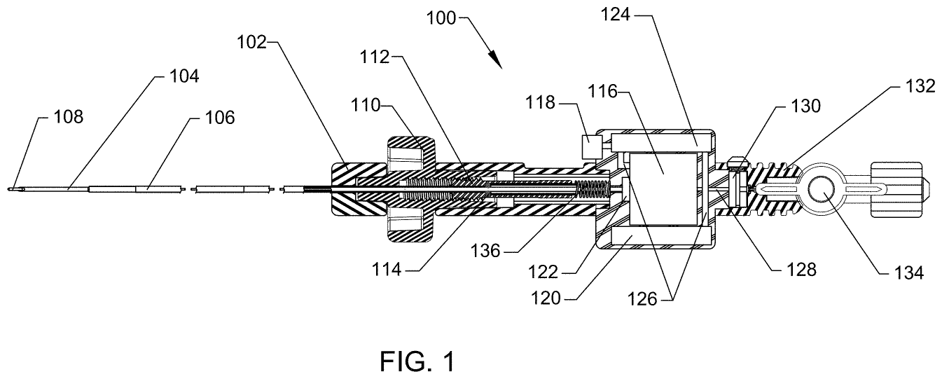

FIG. 1 illustrates a transseptal introducer system 100 with endoluminal punch, comprising a hub 102, an O-ring 114, a jackscrew 112, a control knob 110, a spring 136, a stylet 108, an inner tube 104, an outer tube 106, an inner tube anchor 130, a hub stopcock coupler 132, a stopcock 134, a flow lumen 128, a vibratory driver 116, a power source 120, a bus 126, a controller 124, a control switch, and a central vibrator shaft 122.

The ultrasonic driver 116 can operate in the subsonic range (less than about 1 Hz) all the way up through the ultrasonic range (5-50 kHz). Power levels in the range of about ½ Watt to about 20 Watts can be used with a preferred range of about 2 Watts to about 6 Watts. In the illustrated embodiment, the endoluminal punch is of the steerable variety wherein the control knob 110 can generate off-center forces at the tip to bend a region of outer tube 106 which is selectively made more bendable than the rest of the shaft. In the illustrated embodiment, the vibratory driver 116 is affixed to the hub 102 which is affixed to the anchor 130. The shaft 122 of the vibratory drive 116, which can comprise a hollow lumen (not shown) to allow for fluid transport therethrough, is affixed to the inner tube 104 such that the inner tube 104 is vibrated back and forth axially a small amount which is transmitted to the tip causing a generally axial but also sideways vibratory motion that can be used to promote tissue incision.

FIG. 1 illustrates embodiments wherein a vibratory device 116 can be affixed to the hub of the steerable endoluminal punch system 100. The vibratory device shaft 122 can oscillate back and forth along the longitudinal axis, in an embodiment. Axial vibratory movement of the hub can be transmitted down the length of steerable endoluminal punch shaft such that the distal tip of the steerable endoluminal punch vibrates back and forth along its longitudinal axis. A translator mechanism can be provided which turns this axial vibration into vibration in a direction lateral to that of the longitudinal axis of the steerable endoluminal punch. The vibratory device can comprise a power source derived from a control console, batteries, a wall outlet, or the like. The vibratory device can be operably connected to the power source using a cable, wiring harness, or the like. In these embodiments, the vibratory device 116 and its power and control subcomponents can be integrated into the hub of a needle system 100.

In these embodiments, the vibratory device 116 is directly or indirectly affixed to the anchor 130 of the inner tube 104, the anchor 130 in turn being affixed to the hub 102. The inner tube 104 is affixed to the shaft 122 of the vibratory device and moves therewith. Thus, the inner tube 104 is separated from the hub 102 by the vibratory structure 116. The vibratory mechanism 116 can comprise a loudspeaker type linear movement device, an off-center weight which is rotated about an axis by a motor, a piezoelectric driver, a pneumatic driver, or the like. These systems, in some embodiments, require an input comprising a sine wave structure, or other, which can be controlled in terms of amplitude and frequency and wave shape. In these embodiments, the anchor can be made to vibrate on axis, thus transmitting the vibrator energy to the distal tip of the device through the inner tube and control rod or rods at the distal portion of the steerable endoluminal punch. Since the inner tube is affixed to the outer tube in an off-center manner near the distal end of the steerable endoluminal punch, the distal end of the steerable endoluminal punch will operably vibrate in a lateral direction to the axis of the steerable endoluminal punch. This vibration (oscillatory) energy can provide assistance in cutting through tissue beyond that possible with a static device. The vibratory transducer can be operably connected to a power supply and controller by means of a cable to a power source external to the hub of the steerable endoluminal punch or the power supply can comprise batteries and signal conditioning apparatus and/or software within the steerable endoluminal punch hub. The frequency of vibration can range from less than 1 Hz to ultrasound frequencies of about 40-kHz or more.

In other embodiments, the vibratory apparatus can be affixed to, and operably connected to, a separate control rod or wire disposed within a lumen of the steerable endoluminal punch and routed from a driver at the proximal end to a point at or near the distal end of the steerable endoluminal punch. This control rod or wire can transmit the energy to the distal end of the steerable endoluminal punch wherein it can be used to drive lateral movement, axial movement, rotary movement, or a combination thereof to facilitate tissue penetration and crossing through a hole larger than that which can be made with the steerable endoluminal punch alone, without the energy assistance. The wire or control rod can be affixed to the distal end of the steerable endoluminal punch in an off-center way to generate lateral tip movement in an oscillatory fashion.

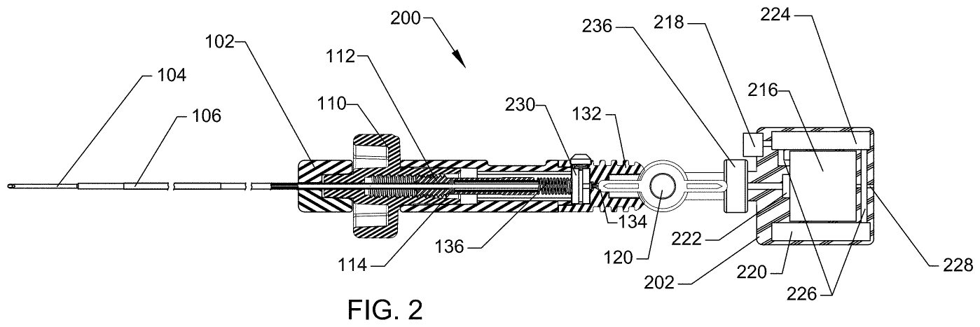

FIG. 2 illustrates another embodiment of the steerable endoluminal punch 200 system wherein the vibratory structure 216 is affixed to the proximal end of the steerable endoluminal punch 200. The steerable endoluminal punch system 200 comprises the inner tube 104, the outer tube 106, the hub 102, the control knob 110, the jackscrew 112, the O-ring 114, the spring 136, the adapter 132, the stopcock 120, and the central lumen 134, which operably communicates between the lumen of the inner tube 104 and the central lumen of the stopcock 120. The steerable endoluminal punch system 200 further comprises an ultrasound driver coupler 236, an ultrasound driver 216 having a central moving shaft 222, a power supply 220, electrical bus 226, a control switch 218, a control system 224, a case 202, and a central lumen 228.

The coupler 236 connects the shaft 222 to the stopcock 120 and therefore the hub 102 of the steerable endoluminal punch. In this configuration, the energy is transmitted through the entire steerable endoluminal punch in an axial fashion but does not cause any relative movement between the inner 104 and outer 106 tubes.

The energized steerable endoluminal punch system 100 and 200 configurations can be used for tissue punch, incision, or penetration, apparatus, etc. They can, in other embodiments, comprise the structure of a guidewire, a stiff track over which other devices or catheters are advanced, an introducer, a catheter, a delivery catheter for an implant or fluids, a therapeutic catheter, a diagnostic catheter, a catheter to support other procedures, or the like. The steerable endoluminal punch can be monitored using fluoroscopy and radiopaque markers affixed or integral to the steerable endoluminal punch. It can also be monitored using ultrasound guidance such as with transesophageal echocardiography, intracardiac echocardiography, real-time three-dimensional echocardiography from transducers and systems affixed to the steerable endoluminal punch.

In other embodiments, vibration can be generated by an ultrasonic transducer mounted within the steerable endoluminal punch tubing. An ultrasonic wire can be disposed along the steerable endoluminal punch tubing through a lumen. An ultrasonic transducer can be affixed to the distal end of the steerable endoluminal punch.

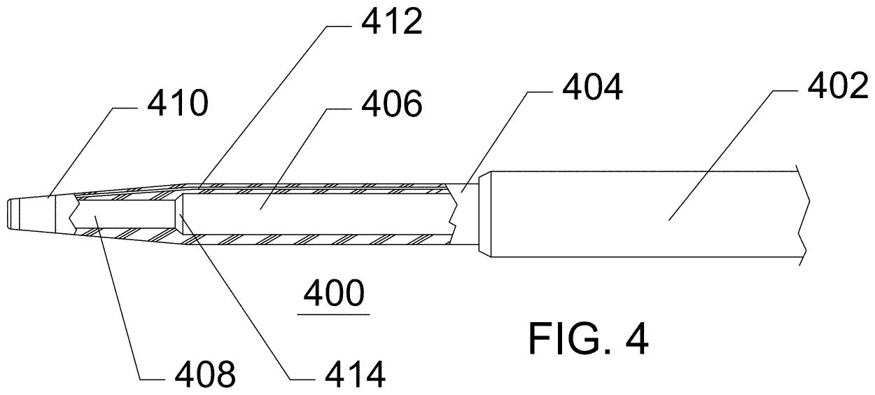

FIG. 4 illustrates a side view, in partial breakaway, of an introducer system 400 comprising a sheath 402, further comprising a central lumen (not shown), and a dilator 404 further comprising a proximal central lumen 406, a stepdown 414, a distal tip lumen 408, and a side lumen 412. In some embodiments, a control linkage (not shown) can be routed through the side lumen 412 such that it is constrained to move axially. Energy drivers, such as 116 and 216 described herein can move the control linkage (not shown) to move elements at the distal end of the dilator 404. In other embodiments, the side lumen 412 can comprise an electrical bus (not shown) which operably connects to an electrode 410 proximate the distal tip of the dilator to generate energy and promote tissue penetration. Such energy generation can comprise modalities such as, but not limited to, microwave radiation, radiofrequency radiation, high frequency focused ultrasound (HIFU), and the like. Energy ranges for the radiofrequency ablation can range from about 2 to about 20 Watts with a preferred range of about 5 to 15 Watts. Energy ranges in the ultrasonic system can range from about 1 Watt to about 20 Watts with a preferred range of about 2 Watts to about 10 Watts.

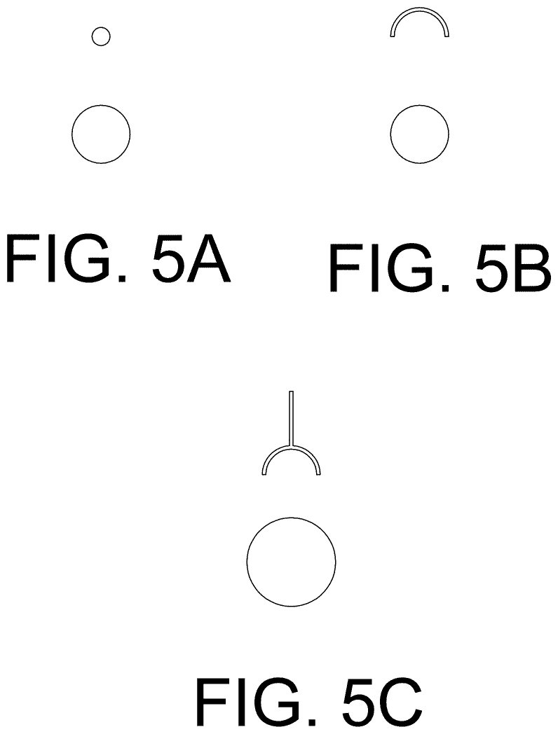

FIG. 5A illustrates the result of using a pointed object to poke a hole in tissue (upper image) and with further configuration to dilate that hole to the size of a transseptal needle, which is 0.032 inches in diameter, as shown in the lower image. The tissue is tightened as it stretches and it does not readily split or incise due to the lack of stress risers resulting from a poke hole being dilated.

FIG. 5B, upper image, illustrates the result of using a half-round blade or half-trephine to cut a semicircular slice in tissue. The trephine can have a flat distal edge or a beveled or other complex shape distal edge. This semicircular slice can fold outward to its full diameter with little or no stress imposed due to dilation, as shown in the bottom image. Additional dilation of this hole can be performed with less force than needed to dilate the hole created in FIG. 5A. The cutting edge of the endoluminal punch can beneficially be described as being formed from a circular tube that is beveled at its distal end. The walls of the endoluminal punch can be sharpened by forming facets or a conical fairing down to a sharp distal edge. If facets are employed, it is possible to generate an extremely sharp tip on the distal edge of the endoluminal punch. One facet on each side can perform cutting of the semicircular tissue incision but it is also beneficially possible to use two or more facets on each side of the cutting edge to maximize sharpness. The facets can be created by grinding, by electron discharge machining (EDM), by laser cutting, by regular machining, or the like. The inner tube that is terminated at its distal end with the sharp structure can be fabricated from 304 stainless steel, 316 stainless steel, or a precipitation hardening stainless steel like 17-7 PH to allow for heat treating and increased strength in these sharp regions.

Note that a stubby blunt stylet, expandable or non-expandable, can be used to shield the sharp pointed distal end of the endoluminal punch (steerable endoluminal punch) from skiving plastic off the wall of the introducer dilator or from getting dulled by the same interaction. It is generally beneficial to align the direction of curvatures of the endoluminal punch with that of the introducer and dilator.

FIG. 5C upper image, illustrates the result of using the half round blade from FIG. 5B but further enhanced with a cutting dilator (FIG. 3A and FIG. 3B) that can generate a slot, shown radially disposed in this FIG. 5B. The radially oriented slot can protrude in various angles to generate maximum tissue splitting with minimal tissue dilation. Thus, larger catheters can pass through the tissue fenestration created by the endoluminal punch used to generate this hole.

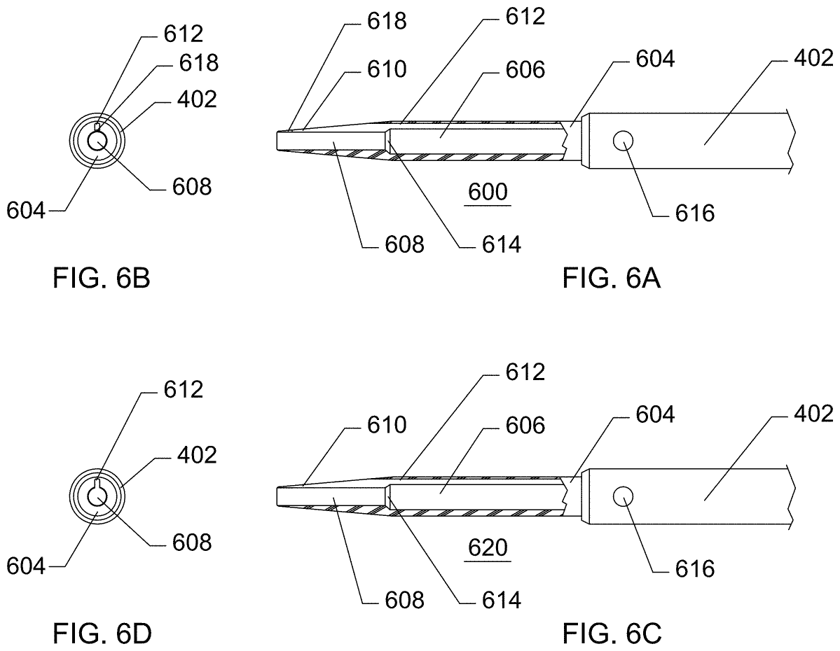

FIG. 6A illustrates a side view, in partial breakaway, of an endoluminal punch introducer 600 comprising a sheath tube 402, further comprising a lumen (not shown) and one or more tip side ports 616. The introducer 600 further comprises a dilator further comprising a dilator shaft 604 further comprising a main lumen 606, a stepdown lumen 608, a stepdown 614, a side projecting lumen 612, a side projecting lumen stop 618, and a tapered tip 610.

The introducer sheath also comprises a proximal end (not shown) further comprising a hub, a hemostasis valve, a side port with a lead line and a stopcock. The introducer sheath tubing 402 can be preferentially reinforced with braided or coil structures fabricated from stainless steel, nitinol, polymeric materials, or the like. The introducer sheath tubing 402 further can comprise a radiopaque marker near its distal end fabricated from materials such as, but not limited to, barium sulfate, bismuth sulfate, tantalum, gold, platinum, platinum iridium, and the like.

The proximal end of the dilator comprises a hub (not shown), further comprising a keyed feature that determines endoluminal punch circumferential orientation. The keyed feature aligns any side projecting elements, including blades, etc., with the side projecting lumen 612 in the dilator shaft 604. The hub (not shown) is affixed to the dilator shaft 604 by adhesive, welding, over-molding or insert molding, swage connectors, and the like.

The side projecting lumen stop 618 can comprise an integral segment of tapered dilator shaft 604, which makes the slotted distal opening to the dilator shaft appear as a window rather than a continuous opening that breaks out in the distal end. The dilator shaft 604 can be reinforced or homogeneous in structure. The dilator shaft 604 should be flexible to varying degrees but comprise functional axial stiffness. The dilator shaft 604 can be extruded with the side projecting lumen integral to the dilator shaft and opening fully into the side of the main lumen 606. The tip 610 can be created or formed by secondary operations such as rf forming, induction forming, and the like. The region where the side projecting lumen 618 breaks through the tapered distal wall 610 can be generated with the mold used for the tip tapering 610 and stepdown 614 in the dilator.

FIG. 6B illustrates an end-on view of the distal end of the endoluminal punch introducer 600 showing the central stepdown lumen 608, the sheath 402, the side projecting lumen 612, the dilator tubing 604, and the side projecting lumen stop 618. The side projecting lumen stop 618 prevents a cutting blade from advancing fully out the distal end of the dilator tapered end 610.

In an embodiment for a standard transseptal introducer, the ID of the dilator main lumen can be approximately 0.055 to 0.065 inches. The diameter of the stepdown lumen can range from about 0.030 inches to about 0.038 inches, depending on the size of instruments intended to be placed therethrough. The stepdown 614 is located at a specific location relative to the distal end to accommodate standard endoluminal punches. The width of the side projecting lumen 612 can range from about 0.005 inches to 0.020 inches. The radial projection of the side projecting lumen 612 can vary to accept specific cutting instruments but in an embodiment can range from about 0.005 to 0.040 inches, depending also on the overall diameter of the dilator tubing.

FIG. 6C illustrates a view, in partial breakaway, of an endoluminal punch introducer 620 comprising a sheath 402, further comprising a lumen (not shown) and one or more tip side ports 616. The side port or ports 616 penetrate completely through the sheath wall 402 and provide fluid communication between the central lumen (not shown) and the ambient environment. The introducer 620 further comprises a dilator, further comprising a dilator shaft 604 further comprising a main lumen 606, a stepdown lumen 608, a stepdown 614, a side projecting lumen 612, and a tapered tip 610.

FIG. 6D illustrates an end-on view of the distal end of the endoluminal punch introducer 620 showing the central stepdown lumen 608, the sheath 402, the side projecting lumen 612, and the dilator tubing 604. In this embodiment, the side projecting lumen 612 cuts completely through to the distal end of the dilator and there is no stop for a side projecting cutting edge.

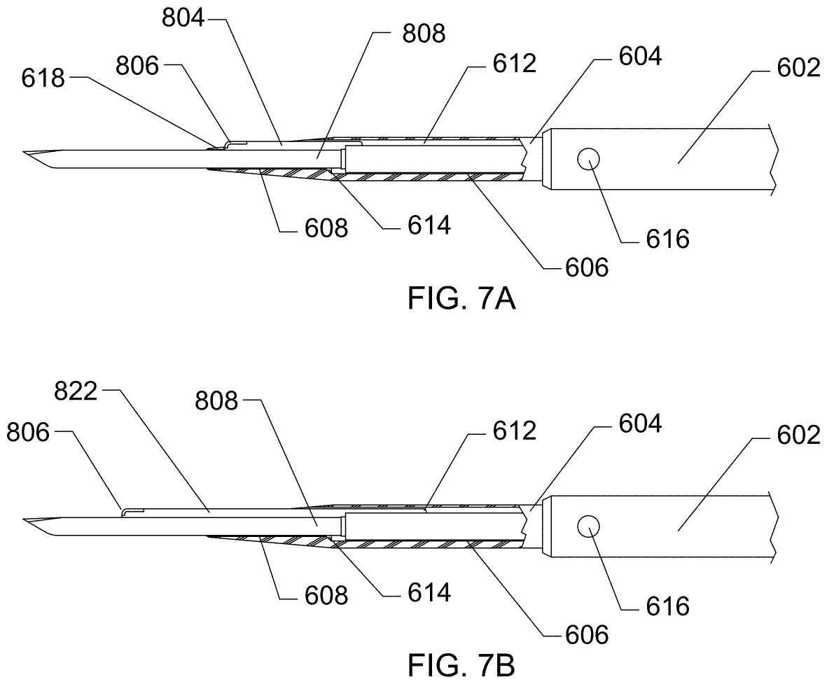

FIG. 7A illustrates a side view, in partial breakaway of the introducer 600 further comprising an endoluminal punch 800 advanced distally against the stepdown 614. The endoluminal punch 800 comprises a shortened side cutting blade 804 with a cutting tip 806. This shortened cutting blade 804 is affixed to the stepdown shaft 808 by methods such as, but not limited to, connectors, adhesives, welding, laser welding, soldering, integral forming, and the like. The cutting tip 806 can be ground or cut by other means on the cutting blade 804 distal end. The distal end of the cutting blade 804 is blocked from unwanted excess distal movement by the cutting blade stop 618. The cutting blade 804 is configured to cut tissue after the tapered tip 610 is partially advanced into the tissue.

FIG. 7B illustrates a side view, in partial breakaway of the introducer 620 with an endoluminal punch 820 inserted therethrough. Note that the proximal end of the cutting blade 804 is extended proximally to achieve greater connection strength with the outer tube 802 of the endoluminal punch. There is no cutting blade stop 618 (refer to FIGS. 6A and 6B) and the cutting blade, elongated in this embodiment, projects completely out the distal end of the tapered dilator tip 610 to cut tissue before it ever reaches the taper 610.

Note that in the case of the devices shown in FIGS. 7A and 7B, retraction of the distal sharp tip 810 of the endoluminal punch 810 results in retraction of the cutting blade 804 within the dilator tip 610, as well, so the system is rendered atraumatic upon endoluminal punch retraction.

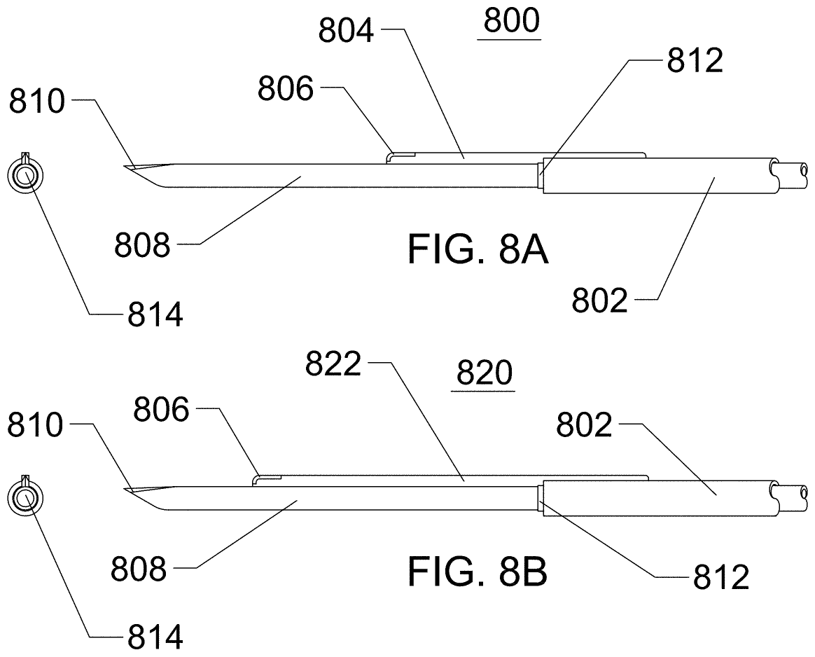

FIG. 8A illustrates the distal end of an endoluminal punch 800 in side exterior view. The endoluminal punch 800 comprises an outer tube 802, an inner tube 808, a weld 812 affixing the outer tube 802 to the inner tube 808, a cutting blade 804 further comprising a sharpened distal end 806, and a sharp distal tip 810 of the inner tube 808. A front view looking at the distal end is also provided showing the central lumen 814 and the distal end of the cutting blade 804. The cutting blade 804 is relatively short and is configured not to extend beyond the distal tip of a dilator. The cutting blade 804 can be affixed to the inner tube 808, the outer tube 802, or both by welding, laser welding, fasteners, integral forming, or the like.

FIG. 8B illustrates the distal end of an endoluminal punch 820 in side exterior view. The endoluminal punch 820 comprises an outer tube 802, an inner tube 808, a weld 812 affixing the outer tube 802 to the inner tube 808, a cutting blade 822 further comprising a sharpened distal end 806, and a sharp distal tip 810 of the inner tube 808. A front view looking at the distal end is also provided showing the central lumen 814 and the distal end of the cutting blade 822. The cutting blade 822 is long and is configured to extend beyond the distal tip of a dilator. The cutting blade 822 can be affixed to the inner tube 808, the outer tube 802, or both by welding, laser welding, fasteners, integral forming, or the like.

Procedurally, this system with the side projecting cutting blade 804 or 822 has advantages in that large incisions can be made in tissue without the need for radiofrequency power, or the like. It is actuated the same way that current transseptal needles are actuated so there is no learning curve for users, although the effects are dramatic, especially in the case where the tissue is scarred, fibrosed, friable, or otherwise damaged. There is, furthermore, no need for a separate cutting mechanism built into the introducer to facilitate passage of the dilator and introducer sheath because the side projecting blade of the endoluminal punch handles this function.

Thus, as described in relation to FIGS. 6A through 8B, the endoluminal punch comprises an elongate member comprising a proximal segment (802) and a distal segment (808), where the distal segment is configured for passage through the dilator and sheath, and the distal segment has a distal tip configured to penetrate body tissue, said distal segment having a cross-section, preferably round, to fit in the dilator. The endoluminal punch also includes a straight cutting blade (804) extending radially from a side of the distal segment. The cutting blade has a sharp edge, suitable for cutting tissue that has been penetrated by the distal tip of the punch. The punch is conveniently formed by two tubes, such that a proximal segment (802) comprises a first tube having a first diameter and a distal segment (808) comprising a tube having a second diameter smaller than the first diameter, and the distal tip of the punch. The distal tip may be configured by penetration of luminal tissue with a sharpened for penetration of body tissue. The sharp edge of the cutting blade may be disposed on a distally facing edge of the cutting blade or a radially facing edge of the cutting blade.

The punch preferably includes a distally facing shoulder formed at a distal end of the first segment, sized to be obstructed by a corresponding proximally facing shoulder in a dilator used to deliver the punch to limit distal translation of the punch distal tip.

The endoluminal punch is configured for use as part of an endoluminal punch system comprising a dilator and a sheath, with the dilator disposed within a lumen of the sheath. The dilator has a distal end and a proximal end and a dilator lumen extending from the proximal end to the distal end, and a channel open the lumen extending from the proximal end to the distal end of the dilator. The proximal segment and the distal segment of the endoluminal punch are configured to be slidably disposed within the dilator lumen, and the cutting blade is sized to be slidably disposed within the channel. Where the punch has a distally facing shoulder, the

The dilator lumen may be formed with a dilator lumen proximal segment and a dilator lumen distal segment, with the dilator lumen proximal segment having a first diameter and the dilator lumen distal segment having a second diameter smaller than the first diameter, such that the dilator lumen distal segment forms a proximally facing shoulder configured to abut the distally facing shoulder at the distal end of the first segment of the endoluminal punch.

The dilator distal segment is preferably formed with a tapered distal tip, and the channel preferably extends radially through the tapered distal tip, such that the cutting blade extends radially from the tapered distal tip when the distal segment of the endoluminal punch is disposed within the tapered distal tip. To limit distal translation of the cutting blade, a cutting blade stop disposed in a distal end of the channel, said cutting blade stop configured.

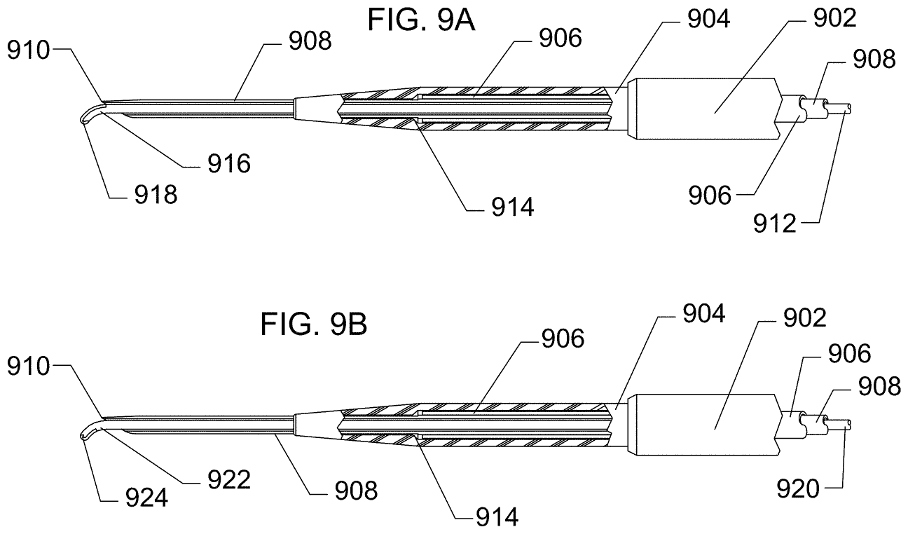

FIG. 9A illustrates a side view of the distal end, in partial cross-section, of an introducer sheath 902 and dilator 904 through which an endoluminal punch is advanced such that the distal end of the outer tube 906 is positioned near its stop point 914, which is a step-down of the inner lumen of the dilator 904. The endoluminal punch comprises an outer tube 906, an inner tube 908 further comprising a sharp tip 910, and a stepdown which is the distal end of the outer tube 906. A piercing stylet 912 is inserted through the central lumen of the inner tube 908. The piercing stylet 912 further comprises a distal curve 916, a sharp cutting edge 918 disposed on the upward and forward facing surfaces of the curved distal end 916.

FIG. 9B illustrates a side view of the distal end, in partial cross-section, of an introducer sheath 902 and dilator 904 through which an endoluminal punch is advanced to a position near its stop point 914. The endoluminal punch comprises an outer tube 906, an inner tube 908 further comprising a sharp tip 910, and a stepdown. A piercing stylet 920 is inserted through the central lumen of the inner tube 908. The piercing stylet 920 further comprises a distal curve 922, a sharp cutting edge 924 disposed on the leading edge and lower trailing edge of the curve 922.

In the illustrated embodiments of FIGS. 9A and 9B, the cutting edges 918 and 924 are integrally formed with the wire region 916 and 922, respectively. The cutting blade structure can also be formed using a separate blade (not shown) that is welded, adhered, affixed using fasteners, and the like to the piercing stylet 912 and 920. In the integrally formed embodiment, as illustrated, the wires (or tubes) 912 and 920 are round or oval and are then curved at the distal end by mechanical means. The curved ends 916 and 922 are then flattened by a press or hammered to form a narrow, flattened, cross-section which is then ground or otherwise cut (EDM, etc.) to form the sharp cutting edges. The cutting edges comprise one or two bevels that are fashioned at angles of about 1 to 25 degrees per side and tapering into a sharp edge. The preferred angles range from about 5 to 15 degrees per side. The wires 912 and 920 can have diameters ranging from about 0.010 inches to about 0.022 inches with a preferred diameter of about 0.012 to 0.016 inches. The smaller diameter allows for easier advancement and more controlled flexing of the curved ends 916 and 922. The curved ends 916 and 922 need to substantially straighten out when the piercing stylet system is inserted into the proximal end of the endoluminal punch, followed by distal advancement through the center lumen of the inner tube 908. Once the curves are exposed beyond the distal end of the inner tube 908, they are free to bias laterally sideways to increase the width of the cutting area of the endoluminal punch system. The piercing stylet system as shown in FIGS. 9A and 9B can be locked to a Luer fitting at the proximal end of the endoluminal punch and can further comprise a spring loaded button to advance and expose the tip beyond the distal end of the endoluminal punch, following which after the spring loaded button is released, the tip retracts automatically within the distal end of the endoluminal punch.

The orientation of the distal curves 916 and 922 can be random or they can be set by a key in the proximal end of the piercing stylet, e.g. hub, and a complimentary keyhole in the hub of the endoluminal punch, or it can be manually oriented using rotational landmarks on the two hubs. Alignment can be set vertically, as illustrated, sideways, or specifically toward the top or bottom of the endoluminal punch tip bevel, etc. In the illustrated embodiment, the blade curves 916 and 922 are offset so that they are pointed approximately oriented 180 degrees from the endoluminal punch point and facets. The cutting edges project sufficiently laterally so as to cut a clinically meaningful incision while being able to be inserted and removed from the endoluminal punch with ease. In the illustrated embodiment, the cutting edge offset is approximately 0.005 to 0.050 inches, or more, beyond the outside diameter of the inner tube 908 with a preferred extent of about 0.010 to 0.030 inches.

The distal curves 916 and 922 form cantilever springs to bias the cutting edges radially outward from the axis of the endoluminal punch. The cutting edges can also be biased using other spring configurations such as coil springs and the like. The distal curves 916 and 922, alone or including the entire piercing stylet wires 912 and 920 can be formed from nitinol and specifically shape memory nitinol so that they spring sideways only after exposure to body temperatures of about 34 to 40 degrees C. These wires can also be superelastic nitinol, cobalt nickel alloy, stainless steel, titanium, PEEK, and the like.

In practice, a piercing stylet or center punch is used when tissue is so difficult to penetrate that the endoluminal punch passes through the tissue but the dilator 904 and the sheath 902 cannot pass through the small incision. The sideways cut of the piercing stylet increases the size of the incision and allows the large diameter sheath and dilator to pass. In some cases, it is beneficial to withdraw an endoluminal punch within the dilator tip if the dilator cannot pass. The piercing stylet is next advanced and exposed distally before the endoluminal punch is exposed out the distal end of the dilator 904. Thus the curve sharp end of the piercing stylet contacts the tissue simultaneously or prior to that of the sharp end 910 of the endoluminal punch.

I claim:

1. An endoluminal punch comprising:
an elongate member comprising a proximal segment (802) and a distal segment (808), said distal segment configured for passage through a dilator and sheath, said distal segment having a distal tip configured to penetrate body tissue, said distal segment having a round cross-section; and
a straight cutting blade extending radially from a side of the distal segment, said cutting blade having a sharp edge.

2. The endoluminal punch of claim 1 wherein:
the proximal segment (802) comprises a tube having a first diameter;
the distal segment (808) comprising a tube having a second diameter smaller than the first diameter; and
the distal tip is sharpened for penetration of body tissue.

3. The endoluminal punch of claim 1 wherein:
the sharp edge is disposed on a distally facing edge of the cutting blade.

4. The endoluminal punch of claim 1 wherein:
the sharp edge is disposed on a radially facing edge of the cutting blade.

5. The endoluminal punch of claim 1 wherein:
the sharp edge is disposed on a radially facing edge of the cutting blade.

6. The endoluminal punch of claim 2 further comprising:
a distally facing shoulder formed at a distal end of the first segment.

7. An endoluminal punch system comprising:
the endoluminal punch of claim 1;
a dilator and a sheath, said dilator disposed within a lumen of the sheath, said dilator having distal end and a proximal end and a dilator lumen extending from the proximal end to the distal end of the dilator, and a channel open the lumen extending from the proximal end to the distal end of the dilator; wherein
the proximal segment and the distal segment are configured to be slidably disposed within the dilator lumen, and said cutting blade is sized to be slidably disposed within the channel.

8. An endoluminal punch system comprising:
the endoluminal punch of claim 6;
a dilator and a sheath, said dilator disposed within a lumen of the sheath, said dilator having distal end and a proximal end and a dilator lumen extending from the proximal end to the distal end of the dilator, and a channel open the lumen extending from the proximal end to the distal end of the dilator; wherein
the proximal segment and the distal segment of the punch are configured to be slidably disposed within the lumen of the dilator, and said cutting blade is sized to be slidably disposed within the channel;
the dilator lumen has a dilator lumen proximal segment and a dilator lumen distal segment, said dilator lumen proximal segment having a first diameter and said dilator lumen distal segment having a second diameter smaller than the first diameter, the dilator lumen distal segment forming a proximally facing shoulder configured to abut the distally facing shoulder at the distal end of the first segment of the endoluminal punch.

9. The endoluminal punch system of claim 7 further wherein:
the dilator distal segment comprises a tapered distal tip, and the channel extends radially through the tapered distal tip, such that the cutting blade extends radially from the tapered distal tip when the distal segment of the endoluminal punch is disposed within the tapered distal tip.

10. The endoluminal punch system of claim 8 further wherein:
the dilator distal segment comprises a tapered distal tip, and the channel extends radially through the tapered distal tip, such that the cutting blade extends radially from the tapered distal tip when the distal segment of the endoluminal punch is disposed within the tapered distal tip.

11. The endoluminal punch system of claim 9 further comprising:
a cutting blade stop disposed in a distal end of the channel, said cutting blade stop configured to limit distal translation of the cutting blade beyond the tapered distal tip.
